(19) United States
(12) Patent Application Publication
(10) Pub. No.: US 2026/0076763 A1
(43) Pub. Date: Mar. 19, 2026

(54) Swappable High Mating Cycle Fiber Connection Interface

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 19/363,380

(22) Filed: Oct. 20, 2025

Related U.S. Application Data
(63) Continuation of application No. 17/945,875, filed on Sep. 15, 2022, now Pat. No. 12,446,988.
(60) Provisional application No. 63/244,992, filed on Sep. 16, 2021.

(51) Int. Cl. A61B 90/00

(52) U.S. Cl. CPC A61B 90/00; A61B 2562/223

(57) ABSTRACT

A medical system is disclosed that includes a medical device having an optical fiber, and an interchangeable connection component configured to provide a fiber optic connection between the medical device and capital equipment. The connection component is configured to facilitate cleaning and/or polishing of fiber optic interfaces include therewith. The connection component is also configured for replacement by a medical technician while the capital equipment is operational. The capital equipment may include one or more of an optical interrogator, a patch cable, a medical probe, an ultrasound machine, a display, a magnet sensor, or an electro-cardiogram (ECG) machine. The medical device may include an elongate member configured for insertion within the patient body, where the optical fiber core extends along a length of the elongate member.

### PRIORITY

This application is a continuation of U.S. patent application Ser. No. 17/945,875, filed Sep. 15, 2022, now U.S. Pat. No. 12,446,988, which claims the benefit of priority to U.S. Provisional Application No. 63/244,992, filed Sep. 16, 2021, each of which is incorporated by reference in its entirety into this application.

### BACKGROUND

The interruption of a medical procedure may put the patient at greater risk and result in an increased cost of providing the procedure. As such, reliability of equipment used in performing medical procedures is very important. Medical equipment may often include capital equipment coupled with a medical device. The capital equipment may be defined for unlimited use (e.g., several hundred up to a few thousand procedures). By way of contrast, the medical device may be defined for limited use, such as a disposable medical device defined for single use. As such, several hundred up to a few thousand medical devices may be connected to and disconnected from the capital equipment which may cause connection components to wear out or fail during the life of the capital equipment. It is not uncommon for the connection components to wear out sooner or fail more often than other components of the capital equipment. As such, resolving the wear out and/or failure propensity of the connection components, may improve reliability and reduce maintenance costs of the capital equipment. Devices and methods, such as those described herein, would reduce patient risk, increase equipment reliability and reduce equipment maintenance costs associated with wear out and failure of the connection components of medical capital equipment.

### SUMMARY OF THE INVENTION

Briefly summarized, disclosed herein is a connection component, including a component body configured for coupling between a medical device and capital equipment, where the medical device includes an optical fiber core having one or more core fibers. A medical device interface is disposed on a first side of the component body, where the medical device interface is configured to operatively couple the component with the medical device, and an equipment interface is disposed on a second side of the component body, where the equipment interface is configured to operatively couple the component with the capital equipment. The component includes one or more optical fibers extending across the component body between the equipment interface and the medical device interface to define a fiber-optic connection between the medical device interface and the equipment interface, and the equipment interface is configured to operatively couple the one or more optical fibers with the capital equipment through a physical barrier of the capital equipment.

The component may include a plurality of optical fibers extending across the component body between the equipment interface and the medical device interface to define a multi-core fiber-optic connection between the medical device interface and the equipment interface. The component may also include one or more electrical conductors extending across the component body between the equipment interface and the medical device interface to define an electrical connection between the medical device interface and the equipment interface.

The component may be configured for cleaning and/or polishing one or more optical fiber interfaces of the medical device interface and/or the equipment interface. The component may also be configured for disinfecting at least a portion of the component.

The component may be configured to facilitate alignment of one or more optical fiber interfaces of the medical device interface with a corresponding one or more optical fiber interfaces of the medical device.

The component may be configured for replacement by a medical clinician, while the equipment is located at a point-of-use, and while the equipment is operational.

Also disclosed herein is a medical system that includes (i) a medical device including an optical fiber, the medical device configured for engagement with a patient body, (ii) capital equipment coupled with the medical device, and (iii) the connection component as summarized above, where the connection component is coupled between the medical device and the capital equipment.

The capital equipment may include one or more of an optical interrogator, a patch cable, a medical probe, an ultrasound machine, a display, a magnet sensor, or an electro-cardiogram (ECG) machine. The medical device may include an elongate member configured for insertion within the patient body, where the optical fiber core extends along a length of the elongate member.

The medical device may include one or more of a catheter, a guidewire, an endoscope, a fiber-optic laser, a pressure sensor, a fluid flow sensor, a blood oxygen sensor, a temperature sensor, or a shape sensor, and the medical device may further include an optical fiber core having a plurality of core fibers.

In some embodiments, the medical device includes one or more electrical wires extending along the length of the elongate member. In use, an electro-cardiogram signal may be transported along the one or more electrical wires and/or the one or more electrical wires may facilitate energizing an electro-magnet of the elongate member.

The medical device may be a disposable device configured for single use. In alternative embodiments, at least a portion of the component may be disinfected and/or sterilized between uses.

Also disclosed herein is a method of performing a medical procedure. The method includes providing a medical system, where the medical system includes a capital equipment portion and a medical device portion that includes an optical fiber core having one or more core fibers. The method further includes (i) decoupling a first connection component of the medical system from the capital equipment portion in response to a wear-out condition of the first connection component, (ii) coupling a second connection component with the capital equipment portion, (iii) coupling the medical device portion with the second connection component, and (iv) operating the medical system to affect the medical procedure.

Decoupling the first connection component and coupling the second connection component may be performed while the medical system is operational and/or by a medical clinician. Coupling the second connection component with the capital equipment portion and coupling the patient interface portion with the second connection component may be performed by the same medical clinician.

The method may further include (i) cleaning an optical fiber interface of the connection component, (ii) polishing an optical fiber interface of the connection component, and/or disinfecting at least a portion of the connection component.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A is an illustrative embodiment of a medical instrument monitoring system including a medical instrument with optic shape sensing and fiber optic-based oximetry capabilities in accordance with some embodiments;

FIG. 1B is an alternative illustrative embodiment of the medical instrument monitoring system 100 in accordance with some embodiments;

FIG. 2 is an exemplary embodiment of a structure of a section of the multi-core fiber core included within the stylet 120 of FIG. 1A in accordance with some embodiments;

FIG. 3A is a first exemplary embodiment of the stylet of FIG. 1A supporting both an optical and electrical signaling in accordance with some embodiments;

FIG. 3B is a cross sectional view of the stylet of FIG. 3A in accordance with some embodiments;

FIG. 4A is a second exemplary embodiment of the stylet of FIG. 1B in accordance with some embodiments;

FIG. 4B is a cross sectional view of the stylet of FIG. 4A in accordance with some embodiments;

FIG. 5A is an elevation view of a first illustrative embodiment of a catheter including integrated tubing, a diametrically disposed septum, and micro-lumens formed within the tubing and septum in accordance with some embodiments;

FIG. 5B is a perspective view of the first illustrative embodiment of the catheter of FIG. 5A including core fibers installed within the micro-lumens in accordance with some embodiments;

FIGS. 6A-6B are flowcharts of the methods of operations conducted by the medical instrument monitoring system of FIGS. 1A-1B to achieve optic 3D shape sensing in accordance with some embodiments;

FIG. 7 is an exemplary embodiment of the medical instrument monitoring system of FIGS. 1A-1B during operation and insertion of the catheter into a patient in accordance with some embodiments;

FIG. 8A is an exploded illustration of a first embodiment of a medical system including a connection component in accordance with some embodiments;

FIG. 8B is a left side perspective view of a second embodiment of the connection component of FIG. 8A in accordance with some embodiments;

FIG. 8C is a right side perspective view of the connection component of FIG. 8B in accordance with some embodiments;

FIG. 8D is a perspective view of another embodiment of the connection component of FIGS. 8B and 8C, in accordance with some embodiments;

FIG. 8E is a side view of a third embodiment of the connection component of FIG. 8A in accordance with some embodiments;

FIG. 9 illustrates a second embodiment of a medical system including a connection component and a display, in accordance with some embodiments;

FIG. 10 illustrates a third embodiment of a medical system including a connection component and ultrasound probe, in accordance with some embodiments; and

FIG. 11 illustrates a fourth embodiment of a medical system including a connection component and magnet sensor, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

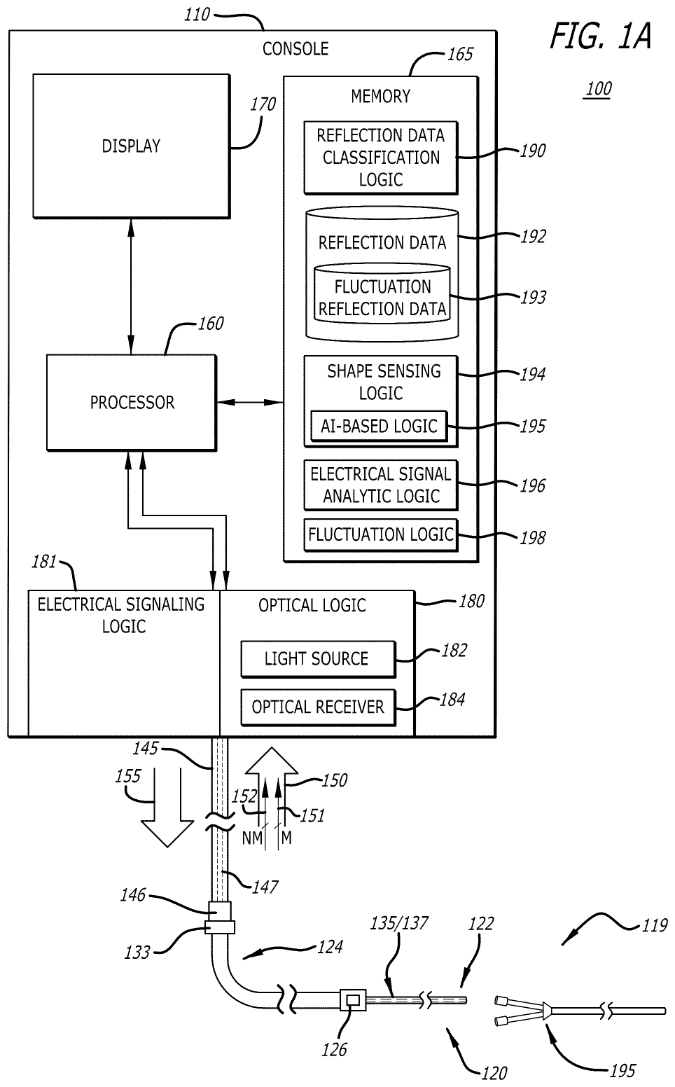

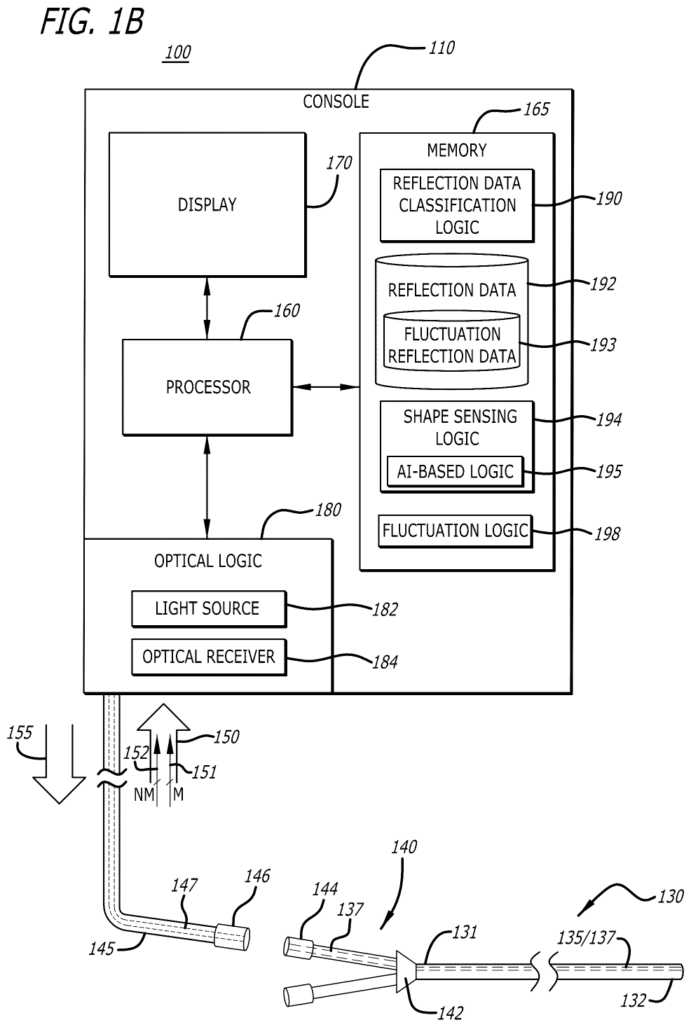

Referring to FIG. 1A, an illustrative embodiment of a medical instrument monitoring system including a medical instrument with optic shape sensing and fiber optic-based oximetry capabilities is shown in accordance with some embodiments. As shown, the system 100 generally includes a console 110 and a stylet assembly 119 communicatively coupled to the console 110. For this embodiment, the stylet assembly 119 includes an elongate probe (e.g., stylet) 120 on its distal end 122 and a console connector 133 on its proximal end 124. The console connector 133 enables the stylet assembly 119 to be operably connected to the console 110 via an interconnect 145 including one or more optical fibers 147 (hereinafter, “optical fiber(s)”) and a conductive medium terminated by a single optical/electric connector 146 (or terminated by dual connectors). Herein, the connector 146 is configured to engage (mate) with the console connector 133 to allow for the propagation of light between the console 110 and the stylet assembly 119 as well as the propagation of electrical signals from the stylet 120 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110.

For both of these embodiments, the content depicted by the display 170 may change according to which mode the stylet 120 is configured to operate: optical, TLS, ECG, or another modality. In TLS mode, the content rendered by the display 170 may constitute a two-dimensional (2D) or three-dimensional (3D) representation of the physical state (e.g., length, shape, form, and/or orientation) of the stylet 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 119, may be used to set the stylet 120 into a desired operating mode and selectively alter operability of the display 170 by the clinician to assist in medical device placement. For example, based on the modality of the stylet 120, the display 170 of the console 110 can be employed for optical modality-based guidance during catheter advancement through the vasculature or TLS modality to determine the physical state (e.g., length, form, shape, orientation, etc.) of the stylet 120. In one embodiment, information from multiple modes, such as optical, TLS or ECG for example, may be displayed concurrently (e.g., at least partially overlapping in time).

Referring still to FIG. 1A, the optical logic 180 is configured to support operability of the stylet assembly 119 and enable the return of information to the console 110, which may be used to determine the physical state associated with the stylet 120 along with monitored electrical signals such as ECG signaling via an electrical signaling logic 181 that supports receipt and processing of the received electrical signals from the stylet 120 (e.g., ports, analog-to-digital conversion logic, etc.). The physical state of the stylet 120 may be based on changes in characteristics of the reflected light signals 150 received at the console 110 from the stylet 120. The characteristics may include shifts in wavelength caused by strain on certain regions of the core fibers integrated within an optical fiber core 135 positioned within or operating as the stylet 120, as shown below. As discussed herein, the optical fiber core 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the stylet 120, and also that of a catheter 195 configured to receive the stylet 120.

According to one embodiment of the disclosure, as shown in FIG. 1A, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the stylet 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 120, and (ii) translate the reflected light signals 150 into reflection data (from repository 192), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from repository 192) to the memory 165 for storage and processing by reflection data classification logic 190. The reflection data classification logic 190 may be configured to: (i) identify which core fibers pertain to which of the received reflection data (from repository 192) and (ii) segregate the reflection data stored with a repository 192 provided from reflected light signals 150 pertaining to similar regions of the stylet 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the stylet 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in 3D space and may further determine the current physical state of the catheter 195 in 3D space for rendering on the display 170.

According to one embodiment of the disclosure, the shape sensing logic 194 may generate a rendering of the current physical state of the stylet 120 (and potentially the catheter 195), based on heuristics or run-time analytics. For example, the shape sensing logic 194 may be configured in accordance with machine-learning techniques to access a data store (library) with pre-stored data (e.g., images, etc.) pertaining to different regions of the stylet 120 (or catheter 195) in which reflected light from core fibers have previously experienced similar or identical wavelength shifts. From the pre-stored data, the current physical state of the stylet 120 (or catheter 195) may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 120 (and/or catheter 195), especially to enable guidance of the stylet 120, when positioned at a distal tip of the catheter 195, within the vasculature of the patient and at a desired destination within the body.

The console 110 may further include electrical signaling logic 181, which is positioned to receive one or more electrical signals from the stylet 120. The stylet 120 is configured to support both optical connectivity as well as electrical connectivity. The electrical signaling logic 181 receives the electrical signals (e.g., ECG signals) from the stylet 120 via the conductive medium. The electrical signals may be processed by electrical signal logic 196, executed by the processor 160, to determine ECG waveforms for display.

Additionally, the console 110 includes a fluctuation logic 198 that is configured to analyze at least a subset of the wavelength shifts measured by sensors deployed in each of the core fibers 137. In particular, the fluctuation logic 198 is configured to analyze wavelength shifts measured by sensors of core fibers 137, where such corresponds to an analysis of the fluctuation of the distal tip of the stylet 120 (or “tip fluctuation analysis”). In some embodiments, the fluctuation logic 198 measures analyzes the wavelength shifts measured by sensors at a distal end of the core fibers 137. The tip fluctuation analysis includes at least a correlation of detected movements of the distal tip of the stylet 120 (or other medical device or instrument) with experiential knowledge comprising previously detected movements (fluctuations), and optionally, other current measurements such as ECG signals. The experiential knowledge may include previously detected movements in various locations within the vasculature (e.g., SVC, Inferior Vena Cava (IVC), right atrium, azygos vein, other blood vessels such as arteries and veins) under normal, healthy conditions and in the presence of defects (e.g., vessel constriction, vasospasm, vessel occlusion, etc.). Thus, the tip fluctuation analysis may result in a confirmation of tip location and/or detection of a defect affecting a blood vessel.

It should be noted that the fluctuation logic 198 need not perform the same analyses as the shape sensing logic 194. For instance, the shape sensing logic 194 determines a 3D shape of the stylet 120 by comparing wavelength shifts in outer core fibers of a multi-core optical fiber to a center, reference core fiber. The fluctuation logic 198 may instead correlate the wavelength shifts to previously measured wavelength shifts and optionally other current measurements without distinguishing between wavelength shifts of outer core fibers and a center, reference core fiber as the tip fluctuation analysis need not consider direction or shape within a 3D space.

In some embodiments, e.g., those directed at tip location confirmation, the analysis of the fluctuation logic 198 may utilize electrical signals (e.g., ECG signals) measured by the electrical signaling logic 181. For example, the fluctuation logic 198 may compare the movements of a subsection of the stylet 120 (e.g., the distal tip) with electrical signals indicating impulses of the heart (e.g., the heartbeat). Such a comparison may reveal whether the distal tip is within the SVC or the right atrium based on how closely the movements correspond to a rhythmic heartbeat.

In various embodiments, a display and/or alert may be generated based on the fluctuation analysis. For instance, the fluctuation logic 198 may generate a graphic illustrating the detected fluctuation compared to previously detected tip fluctuations and/or the anatomical movements of the patient body such as rhythmic pulses of the heart and/or expanding and contracting of the lungs. In one embodiment, such a graphic may include a dynamic visualization of the present medical device moving in accordance with the detected fluctuations adjacent to a secondary medical device moving in accordance with previously detected tip fluctuations. In some embodiments, the location of a subsection of the medical device may be obtained from the shape sensing logic 194 and the dynamic visualization may be location-specific (e.g., such that the previously detected fluctuations illustrate expected fluctuations for the current location of the subsection). In alternative embodiments, the dynamic visualization may illustrate a comparison of the dynamic movements of the subsection to one or more subsections moving in accordance with previously detected fluctuations of one or more defects affecting the blood vessel.

According to one embodiment of the disclosure, the fluctuation logic 198 may determine whether movements of one or more subsections of the stylet 120 indicate a location of a particular subsection of the stylet 120 or a defect affecting a blood vessel and, as a result, of the catheter 130, based on heuristics or run-time analytics. For example, the fluctuation logic 198 may be configured in accordance with machine-learning techniques to access a data store (library) with pre-stored data (e.g., experiential knowledge of previously detected tip fluctuation data, etc.) pertaining to different regions (subsections) of the stylet 120. Specifically, such an embodiment may include processing of a machine-learning model trained using the experiential knowledge, where the detected fluctuations serve as input to the trained model and processing of the trained model results in a determination as to how closely the detected fluctuations correlate to one or more locations within the vasculature of the patient and/or one or more defects affecting a blood vessel.

In some embodiments, the fluctuation logic 198 may be configured to determine, during run-time, whether movements of one or more subsections of the stylet 120 (and the catheter 130) indicate a location of a particular subsection of the stylet 120 or a defect affecting a blood vessel, based on at least (i) resultant wavelength shifts experienced by the core fibers 137 within the one or more subsections, and (ii) the correlation of these wavelength shifts generated by sensors positioned along different core fibers at the same cross-sectional region of the stylet 120 (or the catheter 130) to previously detected wavelength shifts generated by corresponding sensors in a core fiber at the same cross-sectional region. It is contemplated that other processes and procedures may be performed to utilize the wavelength shifts as measured by sensors along each of the core fibers 137 to render appropriate movements in the distal tip of the stylet 120 and/or the catheter 130.

Referring to FIG. 1B, an alternative exemplary embodiment of a medical instrument monitoring system 100 is shown. Herein, the medical instrument monitoring system 100 features a console 110 and a medical instrument 130 communicatively coupled to the console 110. For this embodiment, the medical instrument 130 corresponds to a catheter, which features an integrated tubing with two or more lumen extending between a proximal end 131 and a distal end 132 of the integrated tubing. The integrated tubing (sometimes referred to as “catheter tubing”) is in communication with one or more extension legs 140 via a bifurcation hub 142. An optical-based catheter connector 144 may be included on a proximal end of at least one of the extension legs 140 to enable the catheter 130 to operably connect to the console 110 via an interconnect 145 or another suitable component. Herein, the interconnect 145 may include a connector 146 that, when coupled to the optical-based catheter connector 144, establishes optical connectivity between one or more optical fibers 147 (hereinafter, “optical fiber(s)”) included as part of the interconnect 145 and core fibers 137 deployed within the catheter 130 and integrated into the tubing. Alternatively, a different combination of connectors, including one or more adapters, may be used to optically connect the optical fiber(s) 147 to the core fibers 137 within the catheter 130. The core fibers 137 deployed within the catheter 130 as illustrated in FIG. 1B include the same characteristics and perform the same functionalities as the core fibers 137 deployed within the stylet 120 of FIG. 1A.

The optical logic 180 is configured to support graphical rendering of the catheter 130, most notably the integrated tubing of the catheter 130, based on characteristics of the reflected light signals 150 received from the catheter 130. The characteristics may include shifts in wavelength caused by strain on certain regions of the core fibers 137 integrated within (or along) a wall of the integrated tubing, which may be used to determine (through computation or extrapolation of the wavelength shifts) the physical state of the catheter 130, notably its integrated tubing or a portion of the integrated tubing such as a tip or distal end of the tubing to read fluctuations (real-time movement) of the tip (or distal end).

More specifically, the optical logic 180 includes a light source 182. The light source 182 is configured to transmit the broadband incident light 155 for propagation over the optical fiber(s) 147 included in the interconnect 145, which are optically connected to multiple core fibers 137 within the catheter tubing. Herein, the optical receiver 184 is configured to: (i) receive returned optical signals, namely reflected light signals 150 received from optical fiber-based reflective gratings (sensors) fabricated within each of the core fibers 137 deployed within the catheter 130, and (ii) translate the reflected light signals 150 into reflection data (from repository 192), namely data in the form of electrical signals representative of the reflected light signals including wavelength shifts caused by strain. The reflected light signals 150 associated with different spectral widths include reflected light signals 151 provided from sensors positioned in the center core fiber (reference) of the catheter 130 and reflected light signals 152 provided from sensors positioned in the outer core fibers of the catheter 130, as described below.

As noted above, the shape sensing logic 194 is configured to compare wavelength shifts measured by sensors deployed in each outer core fiber at the same measurement region of the catheter (or same spectral width) to the wavelength shift at the center core fiber positioned along central axis and operating as a neutral axis of bending. From these analytics, the shape sensing logic 190 may determine the shape the core fibers have taken in 3D space and may further determine the current physical state of the catheter 130 in 3D space for rendering on the display 170.

According to one embodiment of the disclosure, the shape sensing logic 194 may generate a rendering of the current physical state of the catheter 130, especially the integrated tubing, based on heuristics or run-time analytics. For example, the shape sensing logic 194 may be configured in accordance with machine-learning techniques to access a data store (library) with pre-stored data (e.g., images, etc.) pertaining to different regions of the catheter 130 in which the core fibers 137 experienced similar or identical wavelength shifts. From the pre-stored data, the current physical state of the catheter 130 may be rendered. Alternatively, as another example, the shape sensing logic 194 may be configured to determine, during run-time, changes in the physical state of each region of the catheter 130, notably the tubing, based on at least (i) resultant wavelength shifts experienced by the core fibers 137 and (ii) the relationship of these wavelength shifts generated by sensors positioned along different outer core fibers at the same cross-sectional region of the catheter 130 to the wavelength shift generated by a sensor of the center core fiber at the same cross-sectional region. It is contemplated that other processes and procedures may be performed to utilize the wavelength shifts as measured by sensors along each of the core fibers 137 to render appropriate changes in the physical state of the catheter 130.

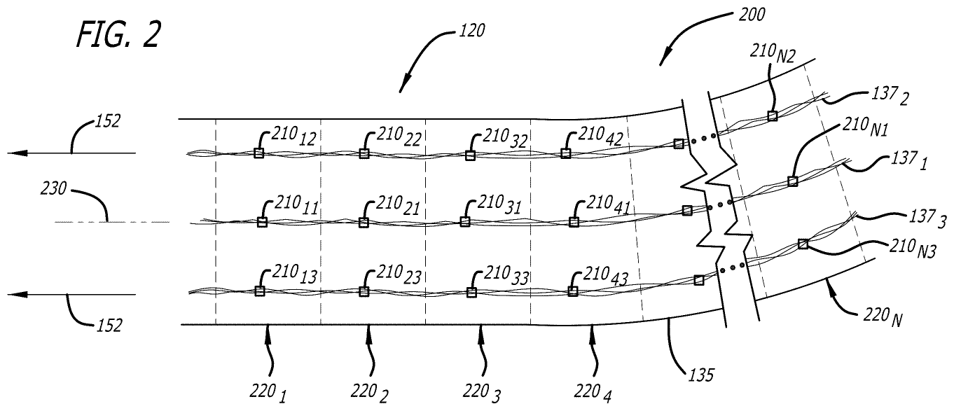

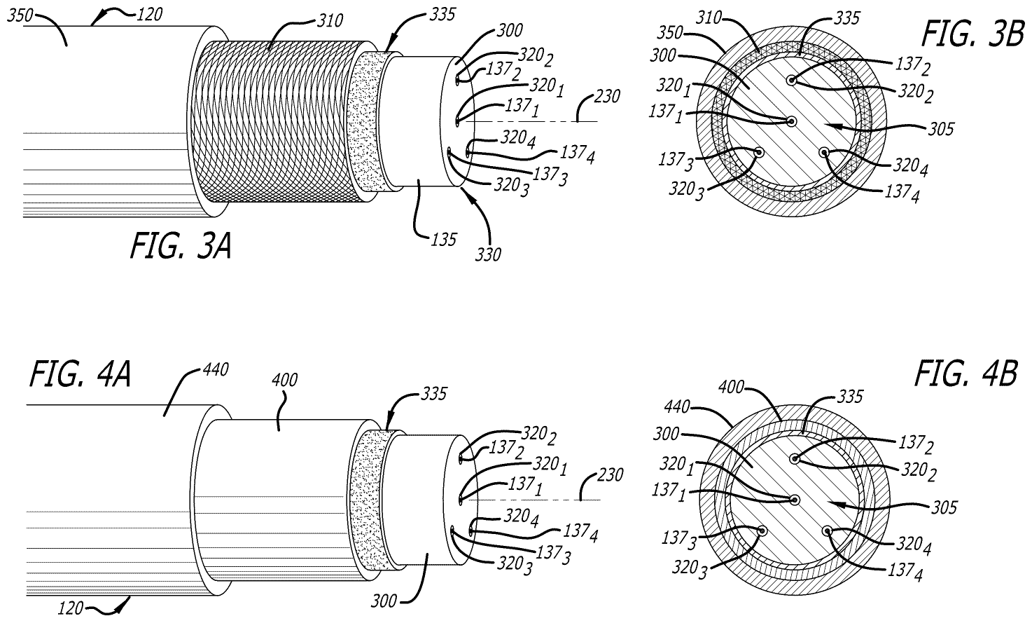

Referring to FIG. 2, an exemplary embodiment of a structure of a section of the multi-core optical fiber included within the stylet 120 of FIG. 1A is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the stylet 120 is operative, each of the reflective gratings 210₁-210_{N} reflects light for a different spectral width. As shown, each of the gratings 210₁ᵢ-210_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the stylet 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the stylet 120 advances in the patient.

For example, with respect to the multi-core optical fiber section 200 of FIG. 2, in response to angular (e.g., radial) movement of the stylet 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the stylet 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the stylet 120. The reflected light signals 150 are reflected back to the console 110 via individual paths over a particular core fiber 137₁-137_{M}.

Referring to FIG. 3A, a first exemplary embodiment of the stylet of FIG. 1A supporting both an optical and electrical signaling is shown in accordance with some embodiments. Herein, the stylet 120 features a centrally located multi-core optical fiber 135, which includes a cladding 300 and a plurality of core fibers 137₁-137_{M} (M≥2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the multi-core optical fiber 135 and the stylet 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the stylet 120, as a greater spacing may provide a lesser rigidity, and thereby, a more pliable stylet 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the stylet 120, the braided tubing 310 provides mechanical integrity to the multi-core optical fiber 135 and operates as a conductive pathway for electrical signals. For example, the braided tubing 310 may be exposed to a distal tip of the stylet 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1B is shown in accordance with some embodiments. Referring now to FIG. 4A, a second exemplary embodiment of the stylet 120 of FIG. 1B supporting both an optical and electrical signaling is shown. Herein, the stylet 120 features the multi-core optical fiber 135 described above and shown in FIG. 3A, which includes the cladding 300 and the first plurality of core fibers 137₁-137_{M} (M≥3; M=4 for embodiment) residing within the corresponding plurality of lumens 320₁-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the stylet 120 in the transfer of electrical signals (e.g., ECG signals) to the console, the conductive tubing 400 may be exposed up to a tip 410 of the stylet 120. For this embodiment of the disclosure, a conductive epoxy 420 (e.g., metal-based epoxy such as a silver epoxy) may be affixed to the tip 410 and similarly joined with a termination/connection point created at a proximal end 430 of the stylet 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

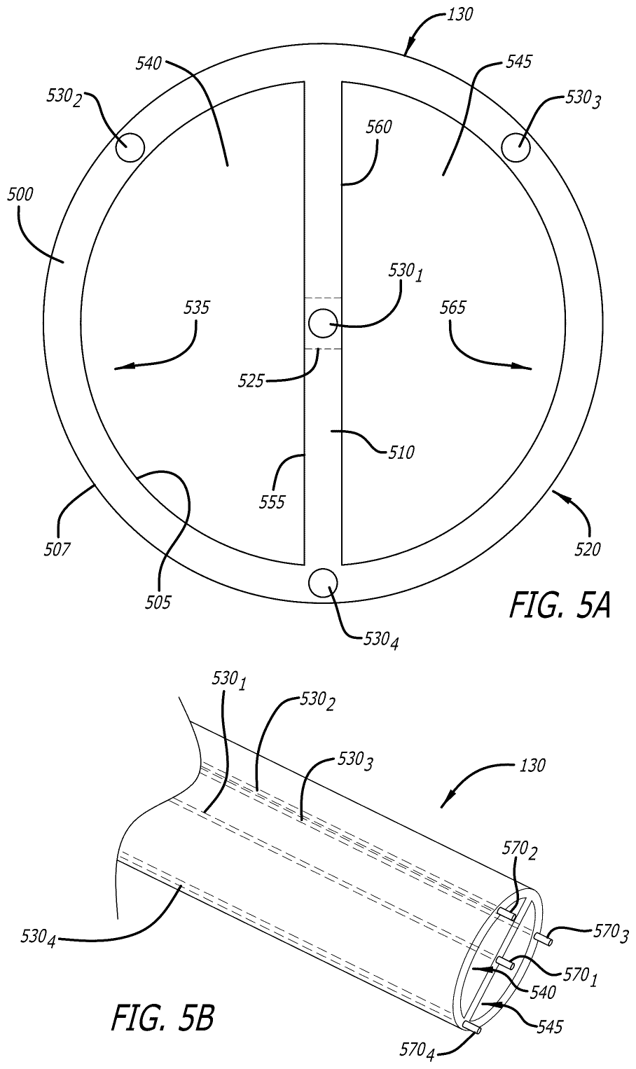

Referring to FIG. 5A, an elevation view of a first illustrative embodiment of a catheter including integrated tubing, a diametrically disposed septum, and micro-lumens formed within the tubing and septum is shown in accordance with some embodiments. Herein, the catheter 130 includes integrated tubing, the diametrically disposed septum 510, and the plurality of micro-lumens 530₁-530₄ which, for this embodiment, are fabricated to reside within the wall 500 of the integrated tubing of the catheter 130 and within the septum 510. In particular, the septum 510 separates a single lumen, formed by the inner surface 505 of the wall 500 of the catheter 130, into multiple lumen, namely two lumens 540 and 545 as shown. Herein, the first lumen 540 is formed between a first arc-shaped portion 535 of the inner surface 505 of the wall 500 forming the catheter 130 and a first outer surface 555 of the septum 510 extending longitudinally within the catheter 130. The second lumen 545 is formed between a second arc-shaped portion 565 of the inner surface 505 of the wall 500 forming the catheter 130 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 130 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 500 of the catheter 130. In particular, a second micro-lumen 530₂ is fabricated within the wall 500 of the catheter 130, namely between the inner surface 505 and outer surface 507 of the first arc-shaped portion 535 of the wall 500. Similarly, the third micro-lumen 5303 is also fabricated within the wall 500 of the catheter 130, namely between the inner and outer surfaces 505/507 of the second arc-shaped portion 555 of the wall 500. The fourth micro-lumen 530₄ is also fabricated within the inner and outer surfaces 505/507 of the wall 500 that are aligned with the septum 510.

According to one embodiment of the disclosure, as shown in FIG. 5A, the micro-lumens 530₂-530₄ are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 130 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 500.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000 μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 130 itself. Similarly, the first micro-lumen 530₁ may be sized to retain the center core fiber 570₁, where the diameter of the first micro-lumen 530₁ may be sized just larger than the diameter of the center core fiber 570₁.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

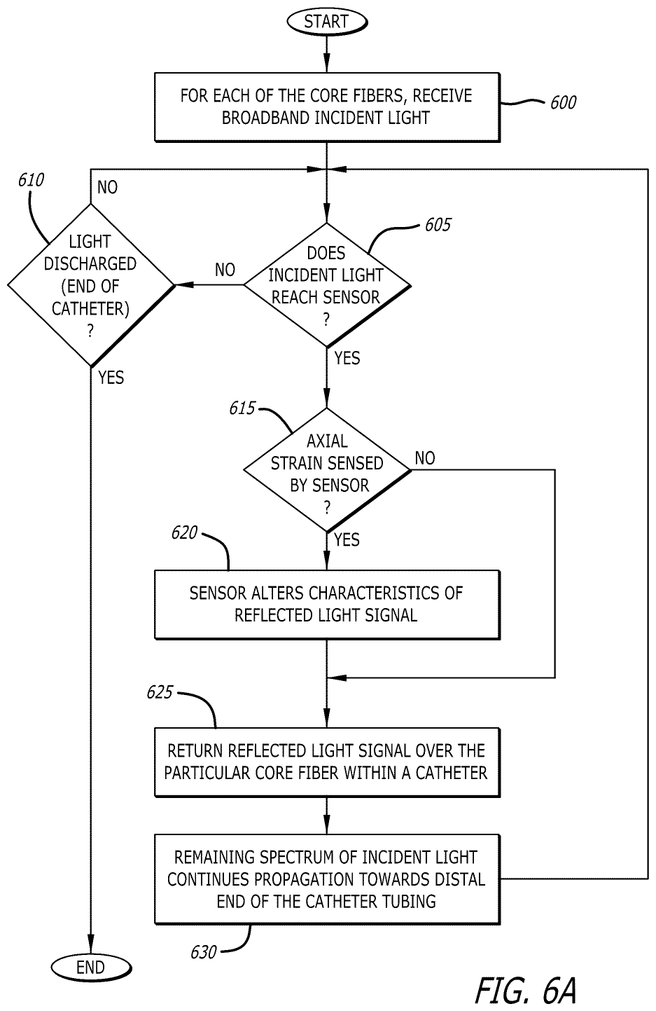

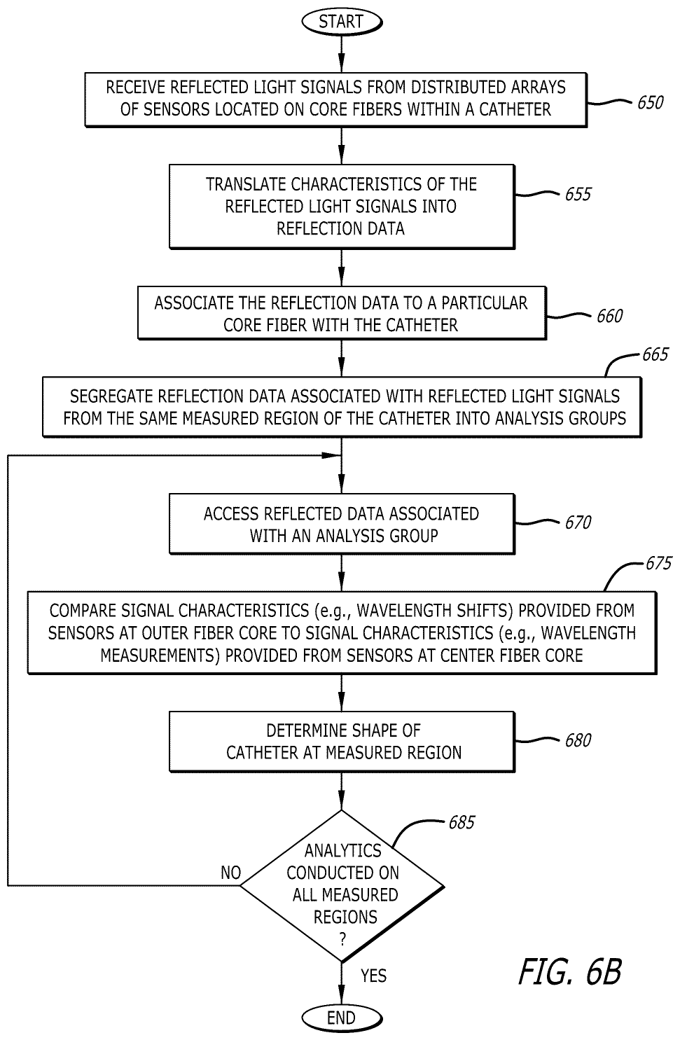

Referring to FIGS. 6A-6B, flowcharts of methods of operations conducted by the medical instrument monitoring system of FIGS. 1A-1B to achieve optic 3D shape sensing are shown in accordance with some embodiments. Herein, the catheter includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter, such as the catheter of FIG. 1B. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From this analytics, on all analytic groups (e.g., reflected light signals from sensors in all or most of the core fibers), the shape sensing logic may determine the shape the core fibers have taken in three-dimensional space, from which the shape sensing logic can determine the current physical state of the catheter in three-dimension space (blocks 680-685).

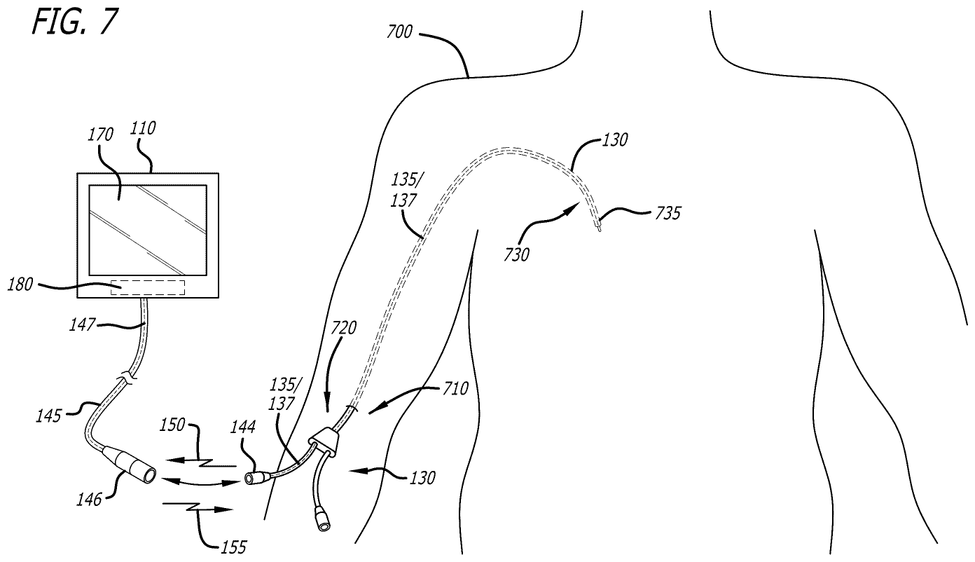

Referring to FIG. 7, an exemplary embodiment of the medical instrument monitoring system of FIG. 1B during operation and insertion of the catheter into a patient are shown in accordance with some embodiments. Herein, the catheter 130 generally includes the integrated tubing of the catheter 130 with a proximal portion 720 that generally remains exterior to the patient 700 and a distal portion 730 that generally resides within the patient vasculature after placement is complete. The (integrated) catheter tubing of the catheter 130 may be advanced to a desired position within the patient vasculature such as a distal end (or tip) 735 of the catheter tubing of the catheter 130 is proximate the patient's heart, such as in the lower one-third (⅓) portion of the Superior Vena Cava (“SVC”) for example. In some embodiments, various instruments may be disposed at the distal end 735 of the catheter 130 to measure pressure of blood in a certain heart chamber and in the blood vessels, view an interior of blood vessels, or the like. In alternative embodiments, such as those that utilize the stylet assembly of FIG. 1A and the catheter 195, such instruments may be disposed at a distal end of the stylet 120.

During advancement through a patient vasculature, the catheter tubing of the catheter 130 receives broadband incident light 155 from the console 110 via optical fiber(s) 147 within the interconnect 145, where the incident light 155 propagates along the core fibers 137 of the multi-core optical fiber 135 within the catheter tubing of the catheter 130. According to one embodiment of the disclosure, the connector 146 of the interconnect 145 terminating the optical fiber(s) 147 may be coupled to the optical-based catheter connector 144, which may be configured to terminate the core fibers 137 deployed within the catheter 130. Such coupling optically connects the core fibers 137 of the catheter 130 with the optical fiber(s) 147 within the interconnect 145. The optical connectivity is needed to propagate the incident light 155 to the core fibers 137 and return the reflected light signals 150 to the optical logic 180 within the console 110 over the interconnect 145. As described below in detail, the physical state of the catheter 130 may be ascertained based on analytics of the wavelength shifts of the reflected light signals 150.

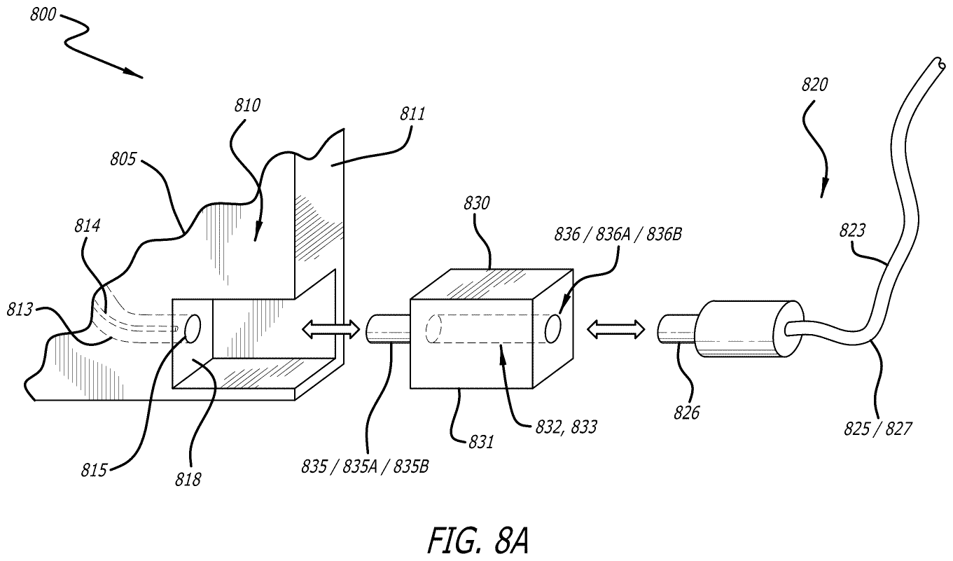

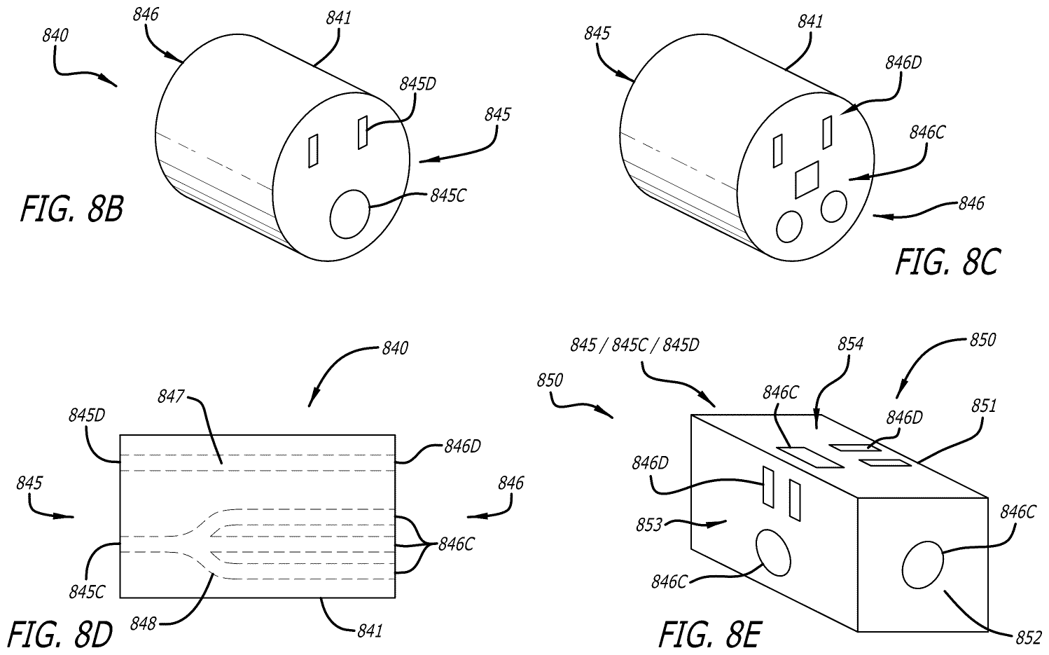

FIG. 8A is an exploded illustration an embodiment of a medical system including a connection component, in accordance with some embodiments. The medical system (system) 800 generally includes a connection component 830, including a component body 831, coupleable between a medical device (device) 820 and capital equipment (equipment) 805. The device 820 includes an optical fiber core 825 having one or more core fibers 827 (e.g., the optical fiber core 135 including the core fibers 137 of FIG. 1A), and the equipment 805 may include a console 810, such as a console resembling in some respects the console 110 of FIG. 1A or 1B, for example. The console 810 is disposed within a housing 811. In use, the connection component 830 is coupled between equipment 805 and the device 820.

The equipment 805 includes an interconnect 813 including one or more optical fibers 847, the interconnect 845 defining a first equipment interface 815. The connection component 830 includes a complementary second equipment interface 835. In use, when the connection component 830 is coupled with the equipment 805, the first equipment interface 815 engages the second equipment interface 835 to facilitate an operative connection, including an optical connection, between the connection component 830 and the equipment 805. For illustrative purposes only, the second equipment interface 835 is shown as a protrusion and the first equipment interface 815 is shown as an opening. However, either or both of the first equipment interface 815 and the second equipment interface 835 may include one or more protrusions and one or more complementary openings to facilitate the operative connection. As will be appreciated by one of ordinary skill, operative connections may include other structural elements such as lateral sliding contact elements, face to face contact elements, or any other suitable connection elements.

The housing 811 includes a barrier wall 818 and the interconnect 845 is disposed inside the barrier wall 818. By way of contrast, the connection component 830 is disposed outside the barrier wall 818. As such, the second equipment interface 835 engages the first equipment interface 815 through the barrier wall 818 when the connection component 830 is coupled with the equipment 805. The barrier wall 818 may define a safety barrier preventing human access to internal electrical components of the equipment 805, thereby preventing a shock hazard.

Similarly, the connection component 830 includes a first device interface 836 configured to receive a complementary second device interface 826 of the device 830. In use, when the device 820 is coupled with the connection component 830, the first device interface 836 engages the second device interface 826 to facilitate an operative connection between the device 820 and connection component 830. By way of summary, coupling the equipment 805 with the connection component 830 and coupling the connection component 830 with the device 820 define an operative connection between the equipment 805 and the device 820.

In some embodiments, the second device interface 826 may be compatible with the first equipment interface 815 so that the device 820 may couple directly with the equipment 805. For example, the equipment 805 and the device 820 may have been originally configured (manufactured) for directly connecting the device 820 to the equipment 805. In such an embodiment, the connection component 830 may be a stand-alone component configured for optional coupling between the equipment 805 and the device 820. In other embodiments, the structure of the equipment interface 815 may prevent coupling of the second device interface 826 therewith (thus, the connection component 830 acts as an adapter that enables a first connection type of the first equipment interface 815 to operatively connect with the device interface 826).

In some embodiments, the component body 831 may define a rectangular box shape or cylindrical shape, having opposite sides. In such embodiments, the second equipment interface 835 and the first device interface 836 may be disposed on opposite sides of the component body 831. In other embodiments, the second equipment interface 835 and the first device interface 836 may be disposed on adjacent sides of the component body 831.

Other shapes of the component body 831 are also contemplated. For example, in some embodiments, the component body 831 may define flexible elongate shape extending between the second equipment interface 835 and first device interface 836 (see FIG. 8B for example). The flexible elongate shape may provide for use of the device 820 at an extended distance away from the equipment 805.

The capital equipment 805 may be configured for unlimited use. In other words, the capital equipment 805 may be configured to perform an unlimited number of medical procedures (e.g., several hundred up to a several thousand procedures). By way of contrast, in some embodiments, the device 820 may be configured for limited use. In some embodiments, the device 820 may be a disposable device configured for performing a single procedure. In other embodiments, the device 820 may be configured for use with a single patient over a relatively small number of procedures (e.g., about one to ten procedures). In still other embodiments, the device 820 may be configured for multiple uses across multiple patients. However, the device 820 may be less durable than the equipment 805 requiring replacement of the device 820 multiple times over the life of the equipment 805. In some embodiments, the device 820 may contact the patient directly during use, thereby requiring disconnection of the device 820 from the equipment 805 for cleaning, disinfection, and/or sterilization between procedures. In short, the equipment 805 may need to accommodate a high number of (e.g., several hundred to several thousand) connection/disconnection cycles with the device 820 over the life of the equipment 805.

The interconnect 813 is disposed within the housing 811 and as such, it may require the services of an equipment repair person to replace the interconnect 813. In other words, in the event of wear out or failure of the first equipment interface 815 of the interconnect 813, the equipment 805 may need to be removed from service so that the equipment repair person may replace the interconnect 813 (i.e., the first equipment interface 815). As service of the equipment 805 may be interrupted, a medical procedure performed by the equipment 805 may also be interrupted and/or delayed.

The connection component 830 may reduce the number of connection/disconnection cycles of the equipment 805 or more specifically the first equipment interface 815 of the interconnect 813. By way of one example, a life of the equipment 805 may include 10,000 procedures employing 10,000 single-use devices 820 resulting in 10,000 coupling/decoupling cycles between the equipment 805 and the device 820. Such an example may also include 100 connection components 830 so that during the life of the equipment 805, the connection component 830 is replaced 100 times. Replacing the connection component 830 100 times defines 100 connection/disconnection cycles between the equipment 805 and the connection component 830 and 100 connection/disconnection cycles between the connection component 830 and the device 820. As such, over the life of the equipment 805, while the equipment 805 is coupled with 10,000 devices 820, the first equipment interface 815 experiences only 100 connection/disconnection cycles.

The device 820 may include an optical fiber core 825 having one or more core fibers 827 for coupling with corresponding fiber-optic components of the console 810, such as the light source 182 or the optical receiver 184 of the console 110 of FIG. 1A, for example. As such, the connection component 830 is configured to operatively couple at least one core fiber 827 of the device 820 with at least one fiber-optic component of the console 810 where broadband light and/or reflected light signals may be optically communicated from the device interface 826 to the equipment interface 815 via one or more core fibers 832. In some embodiments, the device 820 may also include one or more electrical conductors 823 (e.g., wires) for operative coupling of electrical sensors (not shown) of the device 820 with the equipment 805, where electrical signals may be electrically communicated via electrical conductors 833 of the connection component 830.

In some instances, it may be beneficial to perform maintenance procedures on the connection component 830. In some instances, the connection component 830 may be operated within a sterile field, and as such, cleaning (including disinfecting and/or sterilizing) of the connection component 830 may be included in general operating procedures of the system 800. In some embodiments, cleaning the connection component 830 may include separating the connection component 830 from the equipment 805.

The connection component 830 may be configured for sterilization before the first use and/or re-sterilization or disinfection between uses. As such, the connection component 830 may be constructed to be compatible with the sterilization processes, such as an autoclaving processes of the clinical facility, for example. The connection component 830 may be formed of materials configured to withstand exposure to high temperatures and/or disinfecting chemicals consistent with sterilization/disinfection processes of the clinical facility.

In some embodiments, the connection component 830 may include alignment features to facilitate proper alignment of optical fiber interfaces. The connection component 830 may include alignment features 835A to facilitate alignment between the equipment 805 and the connection component 830 and alignment features 836A to facilitate alignment between the connection component 830 and the device 820. The alignment features may include slots, ridges, depressions, protrusions, tapered portions, cross-sectional shapes or any other suitable features to facilitate positional and/or rotational alignment.

In some embodiments, the connection component 830 may include latching/retainment features 835B, 836B to inhibit inadvertent separation of the connection component 830 from the equipment 805 and the device 820, respectively. The latching features may include interference fits, detents, snap fits, hooks, or any other suitable features to inhibit separation of the connection component 830 from the equipment 805 and the device 820. In some embodiments, the latching/retainment features 835B, 836B may include providing tactile feedback to the clinician upon coupling of the connection component 830 with the equipment 805 and/or the device 820.

The connection component 830 includes the second equipment connection interface 835 being an optical fiber interface. It is noted that FIG. 8A includes a circular-shaped optical interface and such is merely a representation of an interface of any optical fiber connector. Thus, the disclosure is not intended to be limited to circular-shaped optical interfaces but may be alternatively shaped depending on the optical fiber connector interface of either the equipment 805 and/or the device 820. For example, the representation of interfaces 835, 836 in FIG. 8A (and FIGS. 8B-8E) is intended to include optical fiber connector interfaces having connector structures of types FC, SC, ST, LC, D4, DIN, MU, etc. The optical fiber interface may be configured for single-mode or multi-mode operation and may also be configured as a single-core or multi-core optical interface.

FIGS. 8B and 8C are left and right side perspective views of a connection component 840, being an alternative embodiment to the connection component 830, illustrating exemplary embodiments of the second equipment interface 845 and first device interface 846, respectively. As shown, in some embodiments, the connection component 840 may be include a cylindrical shape. The second equipment interface 845 includes a single optical interface 845C and the first device interface 846 includes one, two, three, or more different optical fiber interfaces 846C, e.g., any one of the types FC, SC, ST, LC, D4, DIN, MU, etc. In some embodiments, one or more optical fiber interfaces 846C may be the same as the single optical interface 845C. In other embodiments, the single optical interface 845C may differ from all of the optical fiber interfaces 846C.

FIG. 8D is a side view of the connection component 840 showing the component body 841 extending between the second equipment interface 845 and the first device interface 846. As shown via hidden lines, single-core or milti-core optical fibers 848 extend across the component body 841 between the single optical fiber interface 845C of the second equipment interface 845 and the each of the different optical fiber interfaces 846C of the first device interface 846. As shown each optical fiber 848 extending away from each optical fiber interface 846C are combined and coupled with the optical fiber 848 extending away from the single optical fiber interface 845C.

In some embodiments, the connection component 840 may be configured for cleaning and/or polishing of the optical fiber interfaces 845C and/or the optical fiber interfaces 846C. More specifically, the connection component 840 may be structured to provide access to the optical fiber interfaces 845C, 846C in accordance with cleaning and/or polishing processes.

As the device 820 may include one or more electrical conductors 823, the connection component 840 may be configured to operatively couple the electrical conductors 824 of the device 820 with electrical components of the console 810. Hence, the connection component 840 may include one or more electrical conductors 847 extending across the component body 831. Each electrical conductor 847 includes an electrical interface 845D at the second equipment interface 845 and corresponding electrical interface 846D at the first device interface 846. As such, the connection component 840 may facilitate electrical operation of the device 820 having one or more electrical conductors 823.

FIG. 8E illustrates another embodiment of a connection component, in accordance with some embodiments. The connection component 850 may in some respects resemble components of the connection components 830, 840 of FIGS. 8A-8D. The connection component 850 also includes multiple device interfaces 851-853 which may in some respects resemble the first device interface 836 of connection component 830 of FIG. 8A or the first device interface 846 of FIG. 8C. For example, the each of the device interfaces 851-853 may include one or more optical fiber interfaces 846C. Similarly, any or all of the device interfaces 851-853 may also include one or more electrical interfaces 846D.

In the illustrated embodiment, the connection component 850 includes three device interfaces (i.e., device interfaces 851-853). In other embodiments, the connection component 850 may include 2, 4, 5, or more device interfaces. The device interfaces may be disposed on sides of the component body 851 of the connection component 850. In some embodiments, each of the device interfaces 852-854 may be disposed on a side of component body 851 separate from the other device interfaces 852-854. In other embodiments, more than one of the device interfaces 852-854 may be disposed on the same side. In some embodiments, the connection component 850 may be configured to rotate with respect to the equipment 805 (FIG. 8A) to facilitate access to the device interfaces 852-854 disposed on different sides.

In similar fashion as described above in the relation to FIG. 8D, each optical fiber interface 846C is coupled with an optical fiber interface 845C via an optical fiber 848 extending through the component body 851 of the connection component 850, and each electrical interface 846D is coupled with an electrical interface 845D via an electrical conductor 847.

Having the multiple device interfaces 852-854, the connection component 850 may be configured to couple with multiple devices 820. In some embodiments, the multiple devices 820 may be of different types (e.g., configured to perform different medical procedures). In other embodiments, the multiple devices 820 may be of the same type. For example, the device interfaces 852-854 may the same optical fiber interface 846C and/or electrical interfaces 846D, so that a device 820 of one type may be coupled with any of the device interfaces 852-854 or so that multiple devices 820 of the one type may be simultaneously coupled with any combination of the device interfaces 852-854. By way of an alternative example, two or more of the device interfaces 852-854 may include optical fiber interfaces 846C and/or electrical interfaces 846D, so that a device 820 of a first type may only be coupled with a first one of the device interfaces 852-854 and a device 820 of a second type may only be coupled with a second one of the device interfaces 852-854. By way of summary, the connection component 840 may facilitate simultaneous or divided performances of multiple different medical procedures via multiple different devices 820 coupled with the equipment 805. Similarly, the connection component 850 may facilitate simultaneous or divided performances of the same medical procedure via multiple devices 820 of the same type coupled with the equipment 805.

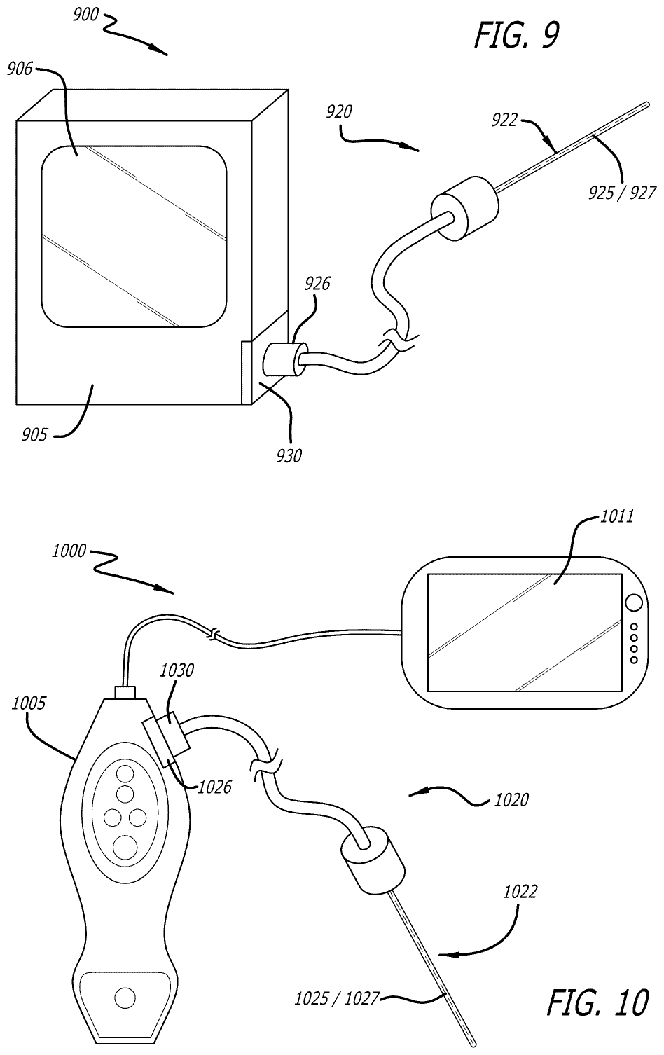

FIG. 9 is an illustration of a second embodiment of a medical system, including a connection component that can, in certain respects, resemble components of the medical system 800 described in connection with FIGS. 8A-8E. It will be appreciated that all the illustrated embodiments may have analogous features. Accordingly, like features are designated with like reference numerals, with the leading digits incremented to “9.” Relevant disclosure set forth above regarding similarly identified features thus may not be repeated hereafter. Moreover, specific features of the medical system 800 and related components shown in FIGS. 8A-8E may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical system of FIG. 9. Any suitable combination of the features, and variations of the same, described with respect to the medical system 800 and components illustrated in FIGS. 8A-8E can be employed with the medical system and components of FIG. 9, and vice versa. This pattern of disclosure applies equally to further embodiments depicted in subsequent figures and described hereafter.

The medical system 900 includes a connection component 930 coupled between equipment 905 having a display 906 and a medical device 920. In some embodiments, the medical device 920 may be include a stylet 922 that in some respects may resemble the stylet 120 of FIG. 1A. The medical device 920 includes an optical fiber core 925 having one or more core fibers 927 extending a long a length of the stylet 922, and the core fibers 927 terminate at a device interface 926 disposed at a proximal end of the medical device 920. The stylet 922 may be configured to define optical signals in accordance with a shape of the stylet 922, and the display 906 may be configured to render images or information pertaining to the shape of the stylet 922 in accordance with the optical signals. The stylet 922 may be configured for insertion within the patient and the optical fiber core 925 may extend along a length of the stylet 922. In some embodiments, the stylet 922 may be configured for insertion within a fluid tract of the patient such as a urinary tract or a vasculature, for example. The medical device 920 may also be configured to obtain, i.e., include sensors (not shown) for obtaining, one or more properties of a patient fluid adjacent the stylet 922, such as a fluid pressure, a fluid flow rate, a fluid temperature, or a blood oxygen level, for example. In some embodiments, the one or more properties may be obtained via the optical fiber core 925. In other embodiments, the optical fiber core 925 may include a fiber-optic laser configured for ablation of body tissue or foreign objects (e.g., a kidney stone).

FIG. 10 is an illustration of a third embodiment of a medical system, including a connection component. The medical system 1000 includes a connection component 1030 coupled between an ultrasound probe 1005 and a medical device 1020 including an optical fiber core 1021. The medical device 1020 may include an elongate instrument 1022 that may in some respects resemble the stylet 922 of FIG. 9 (or may otherwise be a catheter having the optical fiber core 1021 integrated into a wall thereof). The elongate instrument 1022 is configured for insertion within a vasculature of a patient. The optical fiber core 1025 having one or more core fibers 1027 is disposed along the elongate instrument 1022 and terminates at a device interface 1026 disposed at a proximal end of the medical device 1020. The optical fiber core 1021 may be configured to define an optical signal in accordance with a shape of the optical fiber core 1021. The ultrasound probe 1005 may be coupled with a display 1011, and the shape of the optical fiber core 1021 may be rendered on the display 1011 along with an ultrasound image obtained by the ultrasound probe 1005.

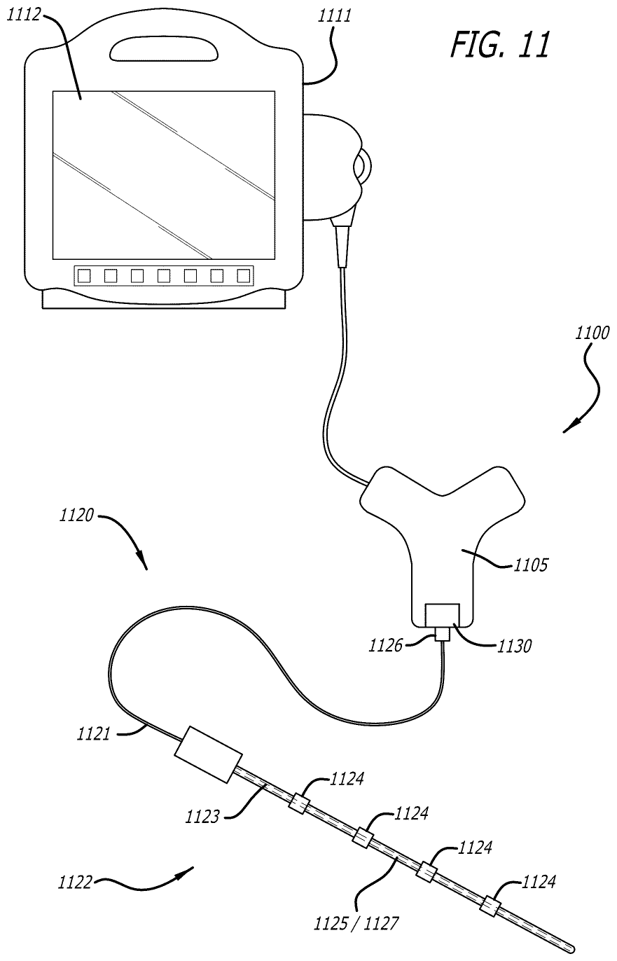

FIG. 11 is an illustration of a fourth embodiment of a medical system, including a connection component. The catheter guidance and tracking system (system) 1100 generally includes a system module 1111, having a display 1112. The system 1100 further includes a magnet sensor 1105 coupled with the system module 1111 and a connection component 1130 coupled between the magnet sensor 1105 and a medical device 1120. The medical device 1120 includes an elongate instrument 1122 that may in some respects resemble the stylet 922 of FIG. 9. The optical fiber core 1125, having one or more core fibers 1127, is disposed along the elongate instrument 1122. The optical fiber core 1121 terminates at a device interface 1126 disposed at a proximal end of the medical device 1120. The elongate instrument 1122 is configured for insertion within a vasculature of a patient, and the optical fiber core 1121 is disposed along the elongate instrument 1122. The optical fiber core 1121 may be configured to define an optical signal in accordance with a shape of the optical fiber core 1121. One or more magnets 1124 are disposed along the elongate instrument 1122. The medical device 1120 may also include one or more electrical conductors 1123 (e.g., wires) extending along the length of the elongate instrument 1122, and in use, at least one electrical conductor 1123 may transport an electro-cardiogram (ECG) signal. In some embodiments, one or more magnets 1124 may be electro-magnets coupled with the magnet sensor 1105 via the electrical conductors 1123.

In use, optical signals from the optical fiber core 1121 together with electrical signals from the electrical conductors 1123 may pass through the connection component 1130 to the magnet sensor 1105. An image rendered on the display 1112 may include the shape of the optical fiber core 1121 along with positions of the magnets 1122 when the elongate instrument 1122 is disposed within and/or is advanced along the vasculature of the patient. The display 1112 may also render images pertaining to the ECG signal.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A method of connecting an optical fiber assembly to capital equipment, comprising:
coupling a connection component to the capital equipment through a physical barrier wall of a housing of the capital equipment, the connection component comprising an equipment interface and one or more optical fibers extending across the connection component in communication with the equipment interface; and
attaching the optical fiber assembly to a medical device interface on the connection component to connect the optical fiber assembly to the capital equipment via the one or more optical fibers in the connection component.

2. The method according to claim 1, wherein the optical fiber assembly comprises a multi-core optical fiber, wherein the connection component includes a plurality of optical fibers extending across the connection component between the equipment interface and the medical device interface to define a multi-core fiber-optic connection between the optical fiber interface and the equipment interface, and wherein attaching the optical fiber assembly to the medical device interface includes connecting the multi-core optical fiber to the capital equipment.

3. The method according to claim 1, wherein the optical fiber assembly includes one or more electrical wires, wherein the connection component includes one or more electrical conductors extending between the equipment interface and the medical device interface, further comprising establishing an electrical connection across the connection component from the optical fiber assembly to the capital equipment.

4. The method according to claim 1, further comprising removing the connection component from the capital equipment, detaching the optical fiber assembly, and sterilizing the connection component.

5. The method according to claim 4, further comprising polishing the connection component.

6. The method according to claim 1, wherein the connection component includes alignment features selected from the group consisting of slots, ridges, depressions, protrusions, tapered portions, cross-sectional shapes, and a combination thereof, further comprising aligning corresponding alignment features on the optical fiber assembly when attaching the optical fiber assembly to the medical device interface.

7. The method according to claim 1, further comprising removing the connection component for replacement while the capital equipment is operational.

8. The method according to claim 1, further comprising disposing the optical fiber assembly after a single use.

9. The method according to claim 1, further comprising viewing output from the optical fiber assembly on a display of the capital equipment.

10. The method according to claim 1, wherein the connection component includes a second medical device interface, further comprising attaching a medical device to the second medical device interface.
